(19) United States
(12) Patent Application Publication
(10) Pub. No.: US 2022/0399122 A1
(43) Pub. Date: Dec. 15, 2022

(54) RISK PREDICTION APPARATUS, RISK PREDICTION METHOD, AND COMPUTER PROGRAM

(71) Applicant: NEC Corporation, Minato-ku, Tokyo (JP)

(72) Inventors: Masahiro HAYASHITANI, Tokyo (JP); Masahiro KUBO, Tokyo (JP)

(73) Assignee: NEC Corporation, Minato-ku, Tokyo (JP)

(21) Appl. No.: 17/771,899

(22) PCT Filed: Nov. 1, 2019
(86) PCT No.: PCT/JP2019/043104

§ 371 Date: Apr. 26, 2022

(51) Int. Cl. G16H 50/30

(52) U.S. Cl. CPC G16H 50/30

(57) ABSTRACT

A risk prediction apparatus includes: an acquisition unit that obtains risk transition data indicating a transition of a risk of deterioration of symptoms from a target patient; an accumulation unit that accumulates the risk transition data of a past about a plurality of patients; a prediction unit that predicts a future change in the risk of the target patient on the basis of the risk transition data about the target patient obtained by the acquisition unit and the risk transition data of the past accumulated in the accumulation unit; and a determination unit that determines whether or not to take a measure for the target patient on the basis of the change in the risk predicted by the prediction unit. This makes it possible to appropriately determine whether or not to take a measure for the patient.

### TECHNICAL FIELD

The present invention relates to a risk prediction apparatus, a risk prediction method, and a computer program that predict a risk of a patient.

### BACKGROUND ART

A known apparatus of this type is an apparatus that predicts a future condition of a patient by using data about the patient (e.g., a patient who is in a hospital, etc.). For example, Patent Literature 1 discloses a technique/technology of predicting a probability of normal tissue complications on the basis of the patient data. Patent Literature 2 discloses a technique/technology of predicting a possibility of developing complications caused by a kidney disease on the basis of a measured value obtained from a test object. Patent Literature 3 discloses a technique/technology of predicting a possibility of complications by using a generated prognostic model.

As another related technique/technology, Patent Literature 4 discloses a technique/technology of proposing the best pharmacotherapy by analyzing data about a patient's history. Patent Literature 5 discloses a technique/technology of calculating a desirable medical treatment condition from a correlation between a biological information about a patient and a condition and a result of a conventional medical treatment.

### CITATION LIST

### Patent Literature

Patent Literature 1: JP2018-514021A
Patent Literature 2: International Publication WO2017/130985 pamphlet
Patent Literature 3: JP2009-533782A
Patent Literature 4: JP2010-020784A
Patent Literature 5: JP2005-267364A

### SUMMARY

### Technical Problem

In the techniques/technologies described in the above Patent Literatures 1 to 3, the future condition of the patient is predicted as a risk of development of complications. In order to suppress the development of the complication, it is required to take an appropriate measure or provide an appropriate treatment (care) for the patient, for example.

However, it is hard to determine whether or not to take a measure for the patient simply by predicting the future condition of the patient. For example, even if the condition of the patient is predicted to deteriorate, it is not easy to make an appropriate decision as to whether an immediate measure should be taken or whether there is no problem even if no measure is taken at the moment. Thus, the respective techniques/technologies described in the Patent Literatures described above have a technical problem that it cannot be appropriately determined whether or not to take a measure for the patient even if the future condition of the patient can be predicted.

The present invention has been made in view of the above problems, and it is an example object of the present invention to provide a risk prediction apparatus, a risk prediction method, and a computer program that are configured to appropriately determine whether or not to take a measure for a patient.

### Solution to Problem

A risk prediction apparatus according to an example aspect of the present invention includes: an acquisition unit that obtains risk transition data indicating a transition of a risk of deterioration of symptoms from a target patient; an accumulation unit that accumulates the risk transition data of a past about a plurality of patients; a prediction unit that predicts a change in the risk of a future of the target patient on the basis of the risk transition data about the target patient obtained by the acquisition unit and the risk transition data of the past accumulated in the accumulation unit; and a determination unit that determines whether or not to take a measure for the target patient on the basis of the change in the risk predicted by the prediction unit.

A risk prediction method according to an example aspect of the present invention obtains risk transition data indicating a transition of a risk of deterioration of symptoms from a target patient; obtains the risk transition data of a past about a plurality of patients; predicts a change in the risk of a future of the target patient on the basis of the risk transition data about the target patient and the risk transition data of the past about the plurality of patients; and determines whether or not to take a measure for the target patient on the basis of the change in the risk predicted.

A computer program according to an example aspect of the present invention obtains risk transition data indicating a transition of a risk of deterioration of symptoms from a target patient; obtains the risk transition data of a past about a plurality of patients; predicts a change in the risk of a future of the target patient on the basis of the risk transition data about the target patient and the risk transition data of the past about the plurality of patients; and determines whether or not to take a measure for the target patient on the basis of the change in the risk predicted.

### Effect of the Invention

According to the risk prediction apparatus, the risk prediction method, and the computer program in the respective aspects described above, it is possible to appropriately determine whether or not to take a measure for a patient on the basis of a change in the risk predicted of the patient.

### BRIEF DESCRIPTION OF DRAWINGS

FIG. 1 is a block diagram illustrating an overall configuration of a risk prediction apparatus according to a first example embodiment.

FIG. 2 is a block diagram illustrating a hardware configuration of the risk prediction apparatus according to the first example embodiment.

FIG. 3 is a flowchart illustrating a flow of operation of the risk prediction apparatus according to the first example embodiment.

FIG. 4 is a graph illustrating an example of risk transition data obtained from a patient.

FIG. 5 is version 1 of a diagram illustrating an example of a method of determining necessity of a measure for the patient.

FIG. 6 is version 2 of a diagram illustrating an example of the method of determining the necessity of a measure for the patient.

FIG. 7 is a block diagram illustrating an overall configuration of a risk prediction apparatus according to a second example embodiment.

FIG. 8 is a flowchart illustrating a flow of operation of the risk prediction apparatus according to the second example embodiment.

### DESCRIPTION OF EXAMPLE EMBODIMENTS

With reference to the drawings, a risk prediction apparatus, a risk prediction method, and a computer program according to example embodiments will be described below.

### First Example Embodiment

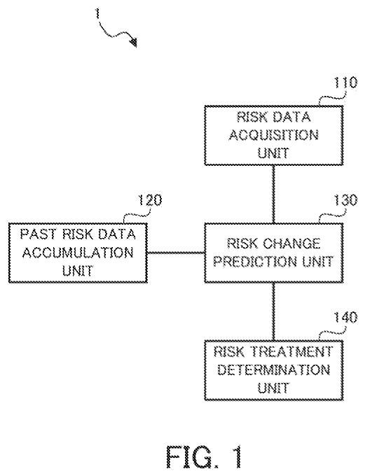

A risk prediction apparatus according to a first example embodiment will be described with reference to FIG. 1 to FIG. 6.

### (Apparatus Configuration)

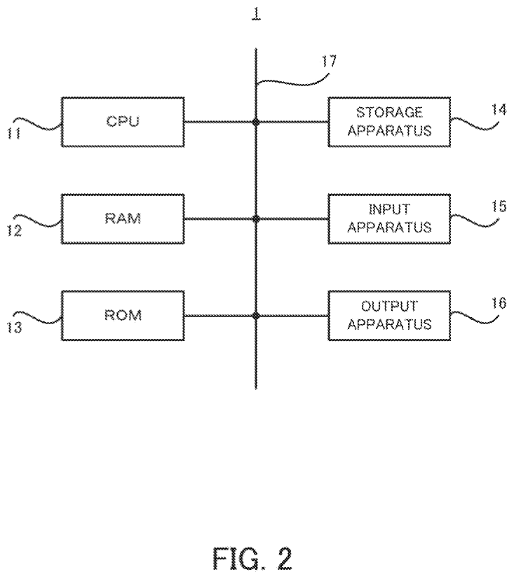

Firstly, with reference to FIG. 1 and FIG. 2, a configuration of the risk prediction apparatus according to the first example embodiment will be described. FIG. 1 is a block diagram illustrating an overall configuration of the risk prediction apparatus according to the first example embodiment. FIG. 2 is a block diagram illustrating a hardware configuration of the risk prediction apparatus according to the first example embodiment.

In FIG. 1, a risk prediction apparatus 1 according to the first example embodiment is an apparatus that predicts a risk of a patient who is in a hospital (specifically, a risk of deterioration of the patient's symptoms) and that determines the necessity of a measure against it. The risk prediction apparatus 1 includes a risk data acquisition unit 110, a past risk data accumulation unit 120, a risk change prediction unit 130, and a risk treatment determination unit 140 as main components.

The risk data acquisition unit 110 is configured to obtain risk transition data indicating a transition of the risk of a target patient, who is a determination target of risk treatment. The risk transition data are an index about a patient condition associated with the risk of deterioration of the patient's symptoms, and can be obtained (or calculated) from not only general vital signs (blood pressure, pulse, body temperature, etc.), but also from FIM (Functional Independence Measure), BI (Barthel Index), NIHSS (National Institute of Health Stroke Scale), MMT (Manual Muscle Test), JCS (Japan Coma Scale), and SpO2 (percutaneous arterial blood oxygen saturation), as well as information about a patient's attributes (e.g., gender, age, etc.). Incidentally, a detailed description pf a specific method of obtaining (or method of calculating) the risk transition data will be omitted here because it is possible to appropriately adopt the existing techniques. The risk transition data obtained by the risk transition data acquisition unit 110 is configured to be outputted to the risk change prediction unit 130.

The past risk data accumulation unit 120 is configured to accumulate the risk transition data obtained in the past (e.g., the risk transition data previously obtained by the risk data acquisition unit 110, or risk data obtained similarly by another apparatus, etc.). The past risk data accumulation unit 120 accumulates the risk transition data not only about the target patient but also about other patients. Furthermore, the past risk data accumulation unit 120 may be configured to collect and share a plurality of risk transition data by using a network or the like. In this case, for example, the past risk data accumulation unit 120 may accumulate the risk transition data collected at one hospital, or may accumulate the risk transition data collected at a plurality of hospitals. The risk transition data of the past accumulated in the past risk data accumulation unit 120 is configured to be outputted to the risk change prediction unit 130, as appropriate.

The risk change prediction unit 130 is configured to predict a change in the risk of the future of the target patient on the basis of the risk transition data about the target patient obtained by the risk data acquisition unit 110 and the risk transition data of the past read from the past risk data accumulation unit 120. A specific method of predicting a change in the risk will be described in detail later. The change in the risk predicted by the risk change prediction unit 130 is configured to be outputted to the risk treatment determination unit 140.

The risk treatment determination unit 140 determines whether or not to take a measure (specifically, a measure to reduce the risk) for the target patient on the basis of the change in the risk of the target patient predicted by the risk change prediction unit 130. A specific determination method by the risk treatment determination unit 140 will be described in detail later. The risk treatment determination unit 140 is configured to output a determination result (i.e., the necessity of a measure) and contents of a measure to a display or the like.

As illustrated in FIG. 2, the risk prediction apparatus 1 according to this example embodiment includes a CPU (Central Processing Unit) 11, a RAM (Random Access Memory) 12, a ROM (Read Only Memory) 13, and a storage apparatus 14. The risk prediction apparatus 1 may also include an input apparatus 15 and an output apparatus 16. The CPU 11, the RAM 12, the ROM 13, the storage apparatus 14, the input apparatus 15, and the output apparatus 16 are connected through a data bus 17.

The CPU 11 reads a computer program. For example, CPU 11 may read a computer program stored by at least one of the RAM 12, the ROM 13 and the storage apparatus 14. For example, the CPU 11 may read a computer program stored by a computer readable recording medium, by using a not-illustrated recording medium read apparatus. The CPU 11 may obtain (i.e., read) a computer program from a not-illustrated apparatus located outside the risk prediction apparatus 1, through a network interface. The CPU 11 controls the RAM 12, the storage apparatus 14, the input apparatus 15, and the output apparatus 16 by executing the read computer program. Especially in this example embodiment, when the CPU 11 executes the read computer program, a functional block for predicting the risk of the target patient and determining whether or not to take a measure is implemented in the CPU 11. The risk data acquisition unit 110, the risk change prediction unit 130, and the risk treatment determination unit 140 described above are implemented, for example, in this CPU 11.

The RAM 12 temporarily stores the computer program to be executed by the CPU 11. The RAM 12 temporarily stores the data that is temporarily used by the CPU 11 when the CPU 11 executes the computer program. The RAM 12 may be, for example, D-RAM (Dynamic RAM).

The ROM 13 stores the computer program to be executed by the CPU 11. The ROM 13 may otherwise store fixed data. The ROM 13 may be, for example, a P-ROM (Programmable ROM).

The storage apparatus 14 stores the data that is stored for a long time by the risk prediction apparatus 1. The storage apparatus 14 may operate as a temporary storage apparatus of the CPU 11. The storage apparatus 14 may include, for example, at least one of a hard disk apparatus, a magneto-optical disk apparatus, an SSD (Solid State Drive), and a disk array apparatus. The past risk data accumulation unit 120 described above may be implemented by the storage apparatus 14.

The input apparatus 15 is an apparatus that receives an input instruction from a user of the risk prediction apparatus 1. The input apparatus 15 may include, for example, at least one of a keyboard, a mouse, and a touch panel. More specifically, the input apparatus 15 may include a smart phone or a tablet owned by a health care professional, a personal computer installed in a hospital, or the like.

The output apparatus 16 is an apparatus that outputs information about the risk prediction apparatus 1 to the outside. For example, the output apparatus 16 may be a display apparatus that is configured to display the information about the risk prediction apparatus 1. More specifically, the output apparatus 16 may be a display of a smart phone or a tablet owned by a healthcare professional, a personal computer installed in a hospital, or the like.

### (Description of Operation)

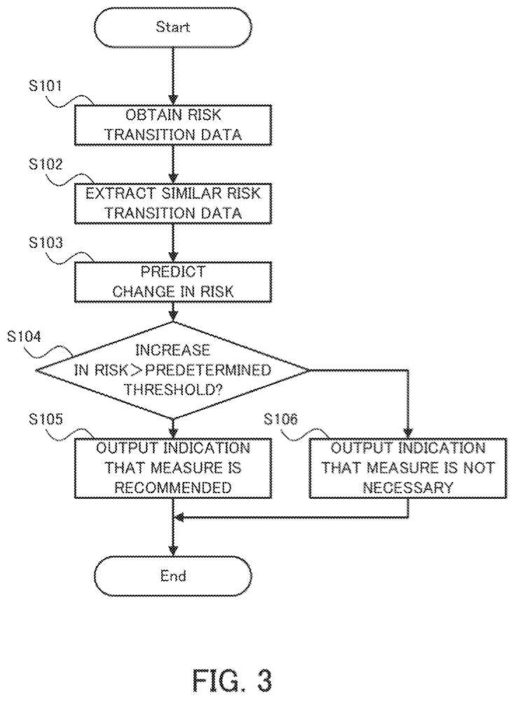

Next, with reference to FIG. 3, a flow of operation of the risk prediction apparatus 1 according to the first example embodiment will be described. FIG. 3 is a flowchart illustrating a flow of the operation of the risk prediction apparatus according to the first example embodiment.

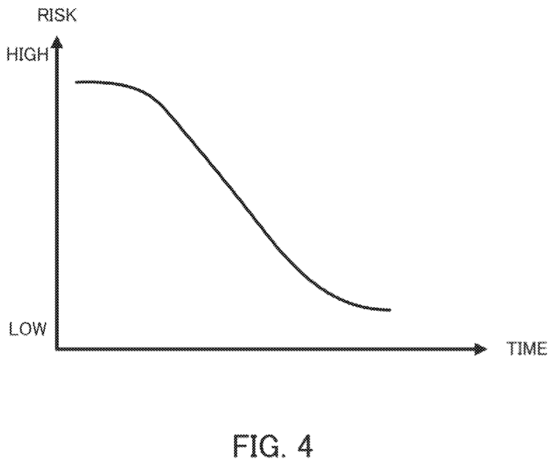

As illustrated in FIG. 3, in operation of the risk prediction apparatus 1 according to the first example embodiment, the risk data acquisition unit 110 firstly obtains the risk transition data about the target patient (step S101). Here, the risk transition data will be specifically described with reference to FIG. 4. FIG. 4 is a graph illustrating an example of the risk transition data obtained from a patient.

As illustrated in FIG. 4, the risk transition data are obtained as data indicating a time change in the risk of the target patient. More specifically, the risk transition data are obtained as data indicating a transition of the risk from a certain timing in the past (e.g., a timing at which the target patient entered a hospital) to the present. For this reason, the risk data acquisition unit 110 may be configured to temporarily store a value of the risk transition data in a certain period. The risk here is a numerical parameter (e.g., a parameter that is larger as the risk is higher and that is smaller as the risk is lower). The risk transition data obtained here are inputted to the risk change prediction unit 130.

Back in FIG. 3, when the risk transition data about the target patient are inputted, the risk change prediction unit 130 extracts the risk transition data of the past from the past risk data accumulation unit 120 (step S102). Specifically, the risk change prediction unit 130 extracts the risk transition data that are similar to the risk transition data about the target patient from among the risk transition data about a plurality of patients accumulated in the past risk data accumulation unit 120. What degree of range is treated as being similar may be determined by setting an optimal parameter through prior simulations, etc. A detailed description of a method of extracting the similar risk transition data is omitted here because the existing technique/technology can be appropriately adopted, but a determination method using a correlation function can be cited as an example.

Subsequently, the risk change prediction unit 130 predicts the change in the risk of the future of the target patient on the basis of the risk transition data about the target patient obtained by the risk data acquisition unit 110 and the risk transition data of the past extracted from the past risk data accumulation unit 120 (step S103). That is, it is predicted how the risk of the target patient will change in the future. The risk of the target patient is predicted, for example, on the assumption of having a similar change as that of the similar past data (e.g., by using a correlation with the past data). Incidentally, a period of predicting a change in the risk may be set in advance; for example, a period corresponding to an expected hospitalization of a patient or the like is set.

Subsequently, the risk treatment determination unit 140 determines whether or not a degree of increase in the risk is greater than or equal to a predetermined threshold on the basis of the change in the risk predicted (step S104). Here, the “degree of increase in the risk” is an index indicating how much the risk is increased, and for example, an increase value or an increase rate of the risk may be used (although a parameter other than the increase value or the increase rate of the risk may be used as the degree of increase in the risk). Furthermore, the “predetermined threshold” is a threshold for determining whether or not to take a measure to reduce the risk for the target patient, and an optimum value is set, for example, in accordance with the risk of occurrence of complications.

When the degree of increase in the risk is greater than or equal to the predetermined threshold (the step S104: YES), the risk treatment determination unit 140 determines that a measure should be taken for the target patient, and outputs an indication that a measure is recommended (step S105). On the other hand, when the degree of increase in the risk is not greater than or equal to the predetermined threshold (the step S104: NO), the risk treatment determination unit 140 determines that it is not necessary to take a measure for the target patient, and outputs an indication that a measure is not necessary (step S106). When it can be determined that a measure should not be taken, an indication that a measure is not recommended may be outputted.

### (Determination of Necessity of Measure)

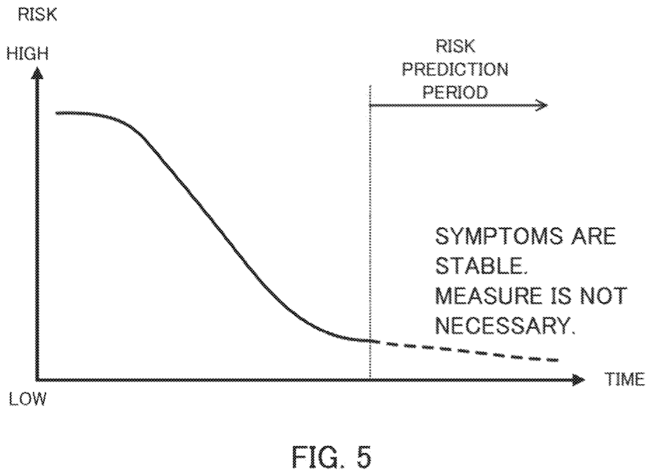

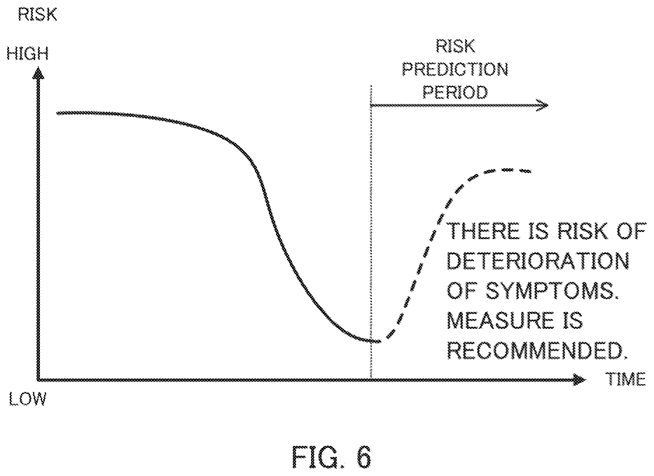

Next, with reference to FIG. 5 and FIG. 6, a specific determination method by the risk treatment determination unit 140 (i.e., the details of the step S104 in FIG. 3) will be described. FIG. 5 is version 1 of a diagram illustrating an example of a method of determining necessity of a measure for the patient. FIG. 6 is version 2 of a diagram illustrating an example of the method of determining the necessity of a measure for the patient.

As illustrated in FIG. 5, when it is predicted that the risk of the target patient will continue to decrease smoothly in the future (see a dashed line in FIG. 5), the degree of increase in the risk will not exceed the predetermined threshold. In this case, the risk treatment determination unit 140 determines that the target patient's symptoms will be stable in the future, and outputs an indication that a measure is not necessary. Alternatively, the risk treatment determination unit 140 may not output information about the measure.

On the other hand, as illustrated in FIG. 6, when it is predicted that the risk of the target patient will increase significantly in the future (see a dashed line in FIG. 6), it is expected that the degree of increase in the risk likely exceeds the predetermined threshold. When the degree of increase in the risk exceeds the predetermined threshold as described above, the risk treatment determination unit 140 determines that the target patient's symptoms will likely deteriorate, and outputs an indication that a measure is recommended. Furthermore, when it is possible to derive a cause of the risk increase from a tendency of the change in the risk (e.g., the occurrence of complications, etc.), the risk treatment determination unit 140 may output information indicating the contents of a measure to reduce the risk. The “information indicating the contents of the measure” here is information specifically indicating what kind of measure should be taken (e.g., information indicating the type, procedure or the like of the measure, etc.).

Incidentally, it is possible to determine the increase in the risk stepwise by setting a plurality of predetermined thresholds. In this case, the information to be outputted may be changed in accordance with the degree of increase in the risk predicted. For example, when the degree of increase in the risk predicted is greater than or equal to a first threshold that is set to be lower, and is less than or equal to a second threshold that is set to be higher (in other words, when the degree of increase in the risk is relatively small), the risk treatment determining section 140 may output an indication that “a measure may be taken,” and when the degree of increase in the risk predicted is greater than or equal to the second threshold that is set to be higher (in other words, when the degree of increase in the risk is relatively large), the risk treatment determining section 140 may output an indication that “a measure should be taken without fail.” Thus, the information indicating the contents of the measure may include information indicating a degree to which the measure may be taken.

Furthermore, when the contents of the measure are outputted, the number and type of measures recommended may be changed in accordance with the degree of increase in the risk. For example, (i) when the predicted risk is greater than or equal to the first threshold that is set to be lower and is less than or equal to the second threshold that is set to be higher, there are less types of measures to be outputted and measures that have a large effect or measures that are easily implemented (e.g., oral care, bed angle up, etc.) are outputted, whereas (ii) when the predicted risk is greater than or equal to the second threshold that is set to be higher, there are more types of measures to be outputted and measures that have a relatively small effect or measures that are effective but are not easily implemented (e.g., breathing exercise, abdominal pressure breathing training, etc.) may be outputted.

### Technical Effect

Next, a technical effect obtained by the risk prediction apparatus 1 according to the first example embodiment will be described.

As described in FIG. 1 to FIG. 6, according to the risk prediction apparatus 1 in the first example embodiment, it is possible to determine whether or not to take a measure for the target patient on the basis of the change in the risk that is predicted from the risk transition data about the target patient and from the risk transition data of the past. It is therefore possible to efficiently prevent the deterioration of the target patient's symptoms (especially, the occurrence of complications).

The occurrence of complications is also a major cause of delayed discharge from a medical facility. Therefore, it is possible to avoid the occurrence of delayed discharge by preventing the occurrence of complications. As a result, beneficial effects can be obtained even for a problem of insufficient number of sickbeds or the like.

A measure to reduce the occurrence of complications may be taken for all the patient, but in that case, a medical staff is required to respond to all the patient, which may significantly increase their workload. In this example embodiment, however, the necessity of a measure is outputted for each patient in accordance with the change in the risk predicted, so that the medical staff can efficiently take a measure for the patient who is to be treated. Therefore, the workload of the medical staff can be reduced.

### Second Example Embodiment

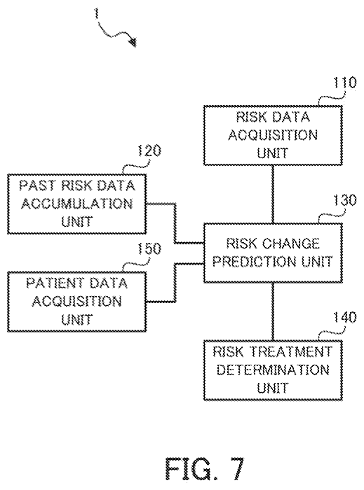

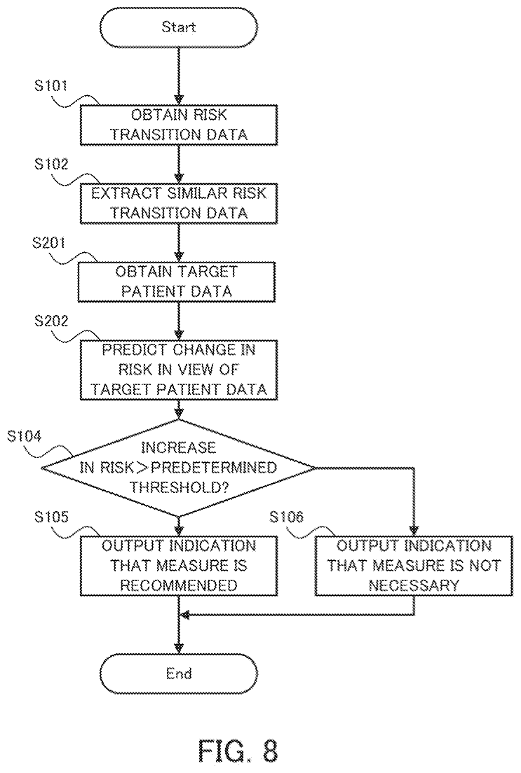

Next, a risk prediction apparatus according to a second example embodiment will be described with reference to FIG. 7 and FIG. 8. The second example embodiment is partially different from the first example embodiment described above only in the configuration and operation, and is substantially the same in the other parts. Therefore, the parts that differ from the first example embodiment described above will be described below, and the other overlapping parts will not be described.

### (Apparatus Configuration)

Firstly, with reference to FIG. 7, a configuration of a risk prediction apparatus 1 according to the second example embodiment will be described. FIG. 7 is a block diagram illustrating an overall configuration of the risk prediction apparatus according to the second example embodiment. Incidentally, in FIG. 7, the same components as those illustrated in FIG. 1 carry the same reference numerals.

As illustrated in FIG. 7, the risk prediction apparatus 1 according to the second example embodiment includes a patient data acquisition unit 150 in addition to the configuration of the first example embodiment (see FIG. 1).

The patient data acquisition unit 150 is configured to obtain target patient data from a target patient. Here, the “target patient data” are data that may affect the change in the risk of the target patient and are different from the risk transition data obtained by the risk data acquisition unit 110 (more specifically, data that are different from various data that are considered as risk data). The target patient data include, for example, information about a medical history of the target patient. The target patient data obtained by the patient data acquisition unit 150 is configured to be outputted to the risk change prediction unit 130.

### (Operation)

### (Description of Operation)

Next, with reference to FIG. 8, a flow of operation of the risk prediction apparatus 1 according to the second example embodiment will be described. FIG. 8 is a flowchart illustrating the flow of the operation of the risk prediction apparatus according to the second example embodiment. Incidentally, in FIG. 8, the same steps as those illustrated in FIG. 3 carry the same reference numerals.

As illustrated in FIG. 8, in operation of the risk prediction apparatus 1 according to the second example embodiment, as in the first example embodiment, the risk data acquisition unit 110 obtains the risk transition data (the step S101), the risk change prediction unit 130 extracts the risk transition data of the past that are similar to the risk transition data about the target patient from the past risk data accumulation unit 120 (the step S102).

Thereafter, in the second example embodiment, the patient data acquisition unit 150 obtains the target patient data from the target patient (step S201). Then, the risk change prediction unit 130 predicts the change in the risk of the target patient, in view of the target patient data obtained by the patient data acquisition unit 150 in addition to the risk transition data about the target patient and the extracted risk transition data of the past (step S202).

The prediction of the change in the risk considering the target patient data makes it possible to predict the risk change of the target patient with higher accuracy than that without considering the target patient data. For example, when the target patient data about the target patient indicate a medical history of complications, then, it can be determined that there is a higher possibility than usual that the target patient will have complications in the future. Thus, in this case, it is predicted that the change in the risk of deterioration of the target patient's symptoms increases, compared to a patient who has no medical history of complications.

Subsequently, the risk treatment determination unit 140 determines whether or not the degree of increase in the risk is greater than or equal to a predetermined threshold on the basis of the change in the risk predicted (the step S104). When the degree of increase in the risk is greater than or equal to the predetermined threshold (the step S104: YES), the risk treatment determination unit 140 outputs an indication that a measure is recommended (the step S105), whereas when the degree of increase in the risk is not greater than or equal to the predetermined threshold (the step S104: NO), the risk treatment determination unit 140 outputs an indication that a measure is not necessary (the step S106).

### Technical Effect

Next, a technical effect obtained by the risk prediction apparatus 1 according to the second example embodiment will be described.

As described in FIG. 7 and FIG. 8, according to the risk prediction apparatus 1 in the second example embodiment, it is possible to more accurately predict the change in the risk of the target patient by using the patient data. As a result, it is possible to more appropriately determine whether or not to take a measure for the patient.

### <Supplementary Notes>

With respect to the example embodiment described above, the following Supplementary Notes will be further disclosed.

### (Supplementary Note 1)

A risk prediction apparatus described in Supplementary Note 1 is a risk prediction apparatus including: an acquisition unit that obtains risk transition data indicating a transition of a risk of deterioration of symptoms from a target patient; an accumulation unit that accumulates the risk transition data of a past about a plurality of patients; a prediction unit that predicts a change in the risk of a future of the target patient on the basis of the risk transition data about the target patient obtained by the acquisition unit and the risk transition data of the past accumulated in the accumulation unit; and a determination unit that determines whether or not to take a measure for the target patient on the basis of the change in the risk predicted by the prediction unit.

### (Supplementary Note 2)

A risk prediction apparatus described in Supplementary Note 2 is the risk prediction apparatus described in Supplementary Note 1, wherein the prediction unit extracts the risk transition data that is similar to the risk transition data obtained by the acquisition unit from a plurality of the risk transition data accumulated in the accumulation unit, and predicts the change in the risk of the future of the target patient on the basis of the risk transition data obtained by the acquisition unit and the extracted risk transition data.

### (Supplementary Note 3)

A risk prediction apparatus described in Supplementary Note 3 is the risk prediction apparatus described in Supplementary Note 2, further including a second acquisition unit that obtains target patient data that is information about the target patient, wherein the prediction unit predicts the change in the risk of the future of the target patient on the basis of the risk transition data obtained by the acquisition unit, the risk transition data accumulated in the accumulation unit, and the target patient data.

### (Supplementary Note 4)

A risk prediction apparatus described in Supplementary Note 4 is the risk prediction apparatus described in Supplementary Note 3, wherein the target patient data include information about a medical history of the target patient.

### (Supplementary Note 5)

A risk prediction apparatus described in any one of Supplementary Notes 1 to 4 is the risk prediction apparatus described in any one o Supplementary Notes 1 to 4, wherein the determination unit determines that the measure should be taken when an increase value or an increase rate of the risk of the future of the target patient predicted by the prediction unit exceeds a predetermined threshold.

### (Supplementary Note 6)

A risk prediction apparatus described in Supplementary Note 6 is the risk prediction apparatus described in any one of Supplementary Notes 1 to 5, wherein the determination unit outputs information indicating contents of the measure when it is determined that the measure should be taken for the target patient.

### (Supplementary Note 7)

A risk prediction apparatus described in Supplementary Note 7 is the risk prediction apparatus described in Supplementary Note 6, wherein the determination unit outputs information indicating contents of each of different measures in accordance with a degree of increase in the risk of the future of the target patient predicted by the prediction unit, when it is determined that the measure should be taken for the target patient.

### (Supplementary Note 8)

A risk prediction apparatus described in Supplementary Note 8 is the risk prediction apparatus described in Supplementary Note 7, wherein the determination unit outputs information indicating contents of each of different types of measures in accordance with an increase value or an increase rate of the risk of the future of the target patient predicted by the prediction unit, when it is determined that the measure should be taken for the target patient.

### (Supplementary Note 9)

A risk prediction apparatus described in Supplementary Note 9 apparatus is the risk prediction apparatus described in Supplementary Note 7 or 8, wherein the determination unit outputs information indicating contents of each of a different number of measures in accordance with an increase value or an increase rate of the risk of the future of the target patient predicted by the prediction unit, when it is determined that the measure should be taken for the target patient.

### (Supplementary Note 10)

A risk prediction apparatus described in Supplementary Note 10 is the risk prediction apparatus described in any one of Supplementary Notes 7 to 9, wherein the determination unit outputs a degree to which the measure should be taken as the information indicating the contents of the measure in accordance with an increase value or an increase rate of the risk of the future of the target patient predicted by the prediction unit, when it is determined that the measure should be taken for the target patient.

### (Supplementary Note 11)

A risk prediction method described in Supplementary Note 11 is a risk prediction method including: obtaining risk transition data indicating a transition of a risk of deterioration of symptoms from a target patient; obtaining the risk transition data of a past about a plurality of patients; predicting a change in the risk of a future of the target patient on the basis of the risk transition data about the target patient and the risk transition data of the past about the plurality of patients; and determining whether or not to take a measure for the target patient on the basis of the change in the risk predicted.

### (Supplementary Note 12)

A computer program described in Supplementary Note 12 is a computer program that allows a computer to operate so as to: obtain risk transition data indicating a transition of a risk of deterioration of symptoms from a target patient; obtain the risk transition data of a past about a plurality of patients; predict a change in the risk of a future of the target patient on the basis of the risk transition data about the target patient and the risk transition data of the past about the plurality of patients; and determine whether or not to take a measure for the target patient on the basis of the change in the risk predicted.

### (Supplementary Note 13)

A recording medium described in Supplementary Note 13 is a recording medium on which the computer program described in Supplementary Note 12 is recorded.

The present invention is not limited to the examples described above and is allowed to be changed, if desired, without departing from the essence or spirit of the invention which can be read from the claims and the entire specification. A risk prediction apparatus, a risk prediction method, and a computer program with such modifications are also intended to be within the technical scope of the present invention.

### DESCRIPTION OF REFERENCE CODES

1 Risk prediction apparatus
11 CPU
12 RAM
13 ROM
14 Storage apparatus
15 Input apparatus
16 Output apparatus
17 Data bus
110 Risk data acquisition unit
120 Past risk data accumulation unit
130 Risk change prediction unit
140 Risk treatment determination unit
150 Patient data acquisition unit

What is claimed is:

1. A risk prediction apparatus comprising:
at least one memory that is configured to store informations; and
at least one processor that is configured to execute instructions
to obtain risk transition data indicating a transition of a risk of deterioration of symptoms from a target patient;
to accumulate the risk transition data of a past about a plurality of patients;
to predict a change in the risk of a future of the target patient on the basis of the risk transition data about the target patient obtained by the acquisition unit and the risk transition data of the past accumulated in the accumulation unit; and
to determine whether or not to take a measure for the target patient on the basis of the change in the risk predicted by the prediction unit.

2. The risk prediction apparatus according to claim 1, wherein the processor is configured to execute instructions to extract the risk transition data that is similar to the risk transition data obtained by the processor from a plurality of the risk transition data accumulated in the processor, and predict the change in the risk of the future of the target patient on the basis of the risk transition data obtained by the processor and the extracted risk transition data.

3. The risk prediction apparatus according to claim 2, wherein the processor is further configured to execute instruction to obtains target patient data that is information about the target patient, wherein
the processor predicts the change in the risk of the future of the target patient on the basis of the risk transition data obtained by the processor, the risk transition data accumulated in the accumulation unit, and the target patient data.

4. The risk prediction apparatus according to claim 3, wherein the target patient data include information about a medical history of the target patient.

5. The risk prediction apparatus according to claim 1, wherein the processor determines that the measure should be taken when an increase value or an increase rate of the risk of the future of the target patient predicted by the processor exceeds a predetermined threshold.

6. The risk prediction apparatus according to claim 1, wherein the processor outputs information indicating contents of the measure when it is determined that the measure should be taken for the target patient.

7. The risk prediction apparatus according to claim 6, wherein the processor outputs information indicating contents of each of different measures in accordance with a degree of increase in the risk of the future of the target patient predicted by the processor, when it is determined that the measure should be taken for the target patient.

8. The risk prediction apparatus according to claim 7, wherein the processor outputs information indicating contents of each of different types of measures in accordance with an increase value or an increase rate of the risk of the future of the target patient predicted by the processor, when it is determined that the measure should be taken for the target patient.

9. The risk prediction apparatus according to claim 7, wherein the processor outputs information indicating contents of each of a different number of measures in accordance with an increase value or an increase rate of the risk of the future of the target patient predicted by the processor, when it is determined that the measure should be taken for the target patient.

10. The risk prediction apparatus according to claim 7, wherein the processor outputs a degree to which the measure should be taken as the information indicating the contents of the measure in accordance with an increase value or an increase rate of the risk of the future of the target patient predicted by the processor, when it is determined that the measure should be taken for the target patient.

11. A risk prediction method comprising:
obtaining risk transition data indicating a transition of a risk of deterioration of symptoms from a target patient;
obtaining the risk transition data of a past about a plurality of patients;
predicting a change in the risk of a future of the target patient on the basis of the risk transition data about the target patient and the risk transition data of the past about the plurality of patients; and
determining whether or not to take a measure for the target patient on the basis of the change in the risk predicted.

12. A non-transitory recording medium on which a computer program is recorded, wherein the computer program that allows a computer to operate so as to:
obtain risk transition data indicating a transition of a risk of deterioration of symptoms from a target patient;
obtain the risk transition data of a past about a plurality of patients;
predict a change in the risk of a future of the target patient on the basis of the risk transition data about the target patient and the risk transition data of the past about the plurality of patients; and
determine whether or not to take a measure for the target patient on the basis of the change in the risk predicted.
